(19) United States
(12) Patent Application Publication
(10) Pub. No.: US 2009/0036917 A1
(43) Pub. Date: Feb. 5, 2009

(54) TOOLS AND METHOD FOR IMPLANTING A SUBCUTANEOUS DEVICE

(76) Inventor: Kenneth M. Anderson, Bloomington, MN (US)

Correspondence Address: MEDTRONIC, INC., 710 MEDTRONIC PARKWAY NE, MINNEAPOLIS, MN, 55432-9924, US

(21) Appl. No.: 11/831,625

(22) Filed: Jul. 31, 2007

(51) Int. Cl. A61B 17/02; A61N 1/362

(52) U.S. Cl. 606/185; 607/5

(57) ABSTRACT

Several tools and simplified implant methods utilizing minimal surgical intrusion into a subject are disclosed for the proper inserting, advancement and positioning of a subcutaneous implantable medical device (IMD). A kit disclosed herein includes a poker for penetrating the skin and generating a small incision, a tissue spreader for increasing the incision width and an introducer and cannula for the proper insertion of the ILR subcutaneously. Diverse IMDs can be implanted using the kit, tools and methods included in the disclosure, including implantable pulse generators (IPGs), implantable loop recorders (ILRs) for collecting and transmitting cardiac activity signals and implantable cardioverter-defibrillators (ICDs) for delivering high voltage cardiac therapy via electrodes spaced from the myocardium.

### FIELD OF THE INVENTION

The present invention generally relates to a minimally invasive implantation of implantable medical devices (IMD); such as, for instance a leadless subcutaneous implantable cardioverter-defibrillator or a cardiac monitor having segmented looping memory for storing physiologic ECG signal events, and the like. The invention is more particularly directed to implant and tunneling tools and methods for the implanting and positioning a subcutaneous ICD or heart monitor in a subject.

### BACKGROUND OF THE INVENTION

Syncopal events and arrhythmias of the heart are particularly problematic for diagnostic physicians to observe in patients. These events, can be of short duration with sudden onset, and may come with little or no warning, while occurring very infrequently. Holter monitors are well known for monitoring electrocardiograms for periods of time amounting to days or perhaps a week, but these are bulky and are applied externally to the body and interfere with the patient's normal life, making them impractical for long-term use. Further, patient compliance cannot always be guaranteed, and is a common problem in using the Holter devices. Problems with external monitors and associated recorders also include inability of some patients to tolerate the attendant skin irritation. Bulky or expensive special purpose devices may need to be available and maintained. Removal is required for showering, and so on. Any time a living body needs to have a long term monitoring of a physiologic event that is intermittent or infrequent or both, all these problems come into focus. Therefore, there exists a need for minimally intrusive long-term monitoring of a patient's physiologic events and status. This is particularly indicated in, but not limited to patients with cardiac arrhythmias and vasovagal syncope to provide sufficient evidence and data for diagnostic purposes to more accurately prescribe treatment (i.e., drugs, pacemakers, defibrillators, and the like) and conduct research into the causes and effects of such events.

Patients have come to accept long term implants of small items for many things including birth control, for example, like the “Norplant” (trademark of Wyeth Laboratories) devices, which secrete birth control hormones for perhaps a year before they need replacing. Similarly, chronic monitoring of cardiac function can provide valuable diagnostic information for a variety of cardiovascular conditions, including arrhythmias, heart failure, syncope or other autonomic system abnormalities. For example, chronic monitoring of the ECG subcutaneously, using a device such as the Reveal® Insertable Loop Recorder (ILR) available from Medtronic, Inc., can be useful in diagnosing infrequent symptomatic episodes such as unexplained syncope and for capturing asymptomatic arrhythmias, which may be of interest to the patient's physician. Accordingly it is believed that small, inexpensively implanted devices for long-term monitoring will be well tolerated by the patient population to be served by this invention.

Accordingly, there exists a need for a more acceptable recording and monitoring device capable to maintain a data record over a long period of time and highlighting or capturing those physiologic events that are of interest to a diagnostic, research or therapeutic study and particularly those physiologic events that are required for correct diagnosis and therapy of life threatening arrhythmias. Further, it has heretofore required a surgeon and implanting staff, the sterile environment of a surgical suite, a cautery machine to close flood flow from the incision(s) and a hospital stay for the patient to implant an ILR in a patient. This has been expensive, limiting the use of the ILR and overly invasive to the patient requiring a subcutaneous implant of intracardiac monitors for simple recording functions.

### SUMMARY OF THE INVENTION

A method and apparatus is described which provides for an improved implantation of a minimally intrusive implantable system capable of communicating with an external device and having subcutaneous electrodes to measure a subcutaneous electrogram. The system includes a signal input means, a looping memory, detection circuitry, a circuit for controlling the memory. Furthermore, the system includes a housing design that enables minimal intrusion and adapted to be implanted using simple implant tools, enabling an efficient method to facilitate the implantation of the device.

These foregoing needs in the art are also reflected in subcutaneous therapy delivery platforms, such as a subcutaneous ICD having high voltage electrodes disposed directly upon the housing of the subcutaneous ICD or having short, or snub-type, lead-based high voltage electrodes operatively coupled to high voltage circuitry disposed within the subcutaneous ICD housing.

Although the depicted embodiments include an ILR the present invention expressly includes all manner of subcutaneous IMDs, and specifically so called subcutaneous ICD adapted to deliver high voltage cardioversion and/or defibrillation therapies.

### BRIEF DESCRIPTION OF THE DRAWINGS

These and other aspects and features of the present invention will be appreciated as the same becomes better understood by reference to the following detailed description of the various embodiments of the invention when considered in connection with the accompanying drawings, in which like numbered reference numbers designate like parts throughout the figures thereof.

FIG. 1 is a frontal view of a patient in whom an ILR may be implanted with a typical location referenced thereon.

FIG. 2 is a schematic block diagram illustrating the main circuit of an ILR in accord with the present invention.

FIG. 3 is a plan view illustrating an apparatus to aid in the implant of an ILR in accordance with the present invention.

FIG. 4A is a plan view illustrating an apparatus to aid in the implant of an ILR in accordance with the present invention.

FIG. 4B is a plan view illustrating an alternative apparatus to aid in the implant of an ILR in accordance with the present invention.

FIG. 5A is a plan view illustrating an apparatus to aid in the implant of an ILR in accordance with the present invention.

FIG. 5B is a plan view illustrating an ILR in accordance with the present invention.

FIG. 6 is a plan view illustrating an apparatus to aid in the implant of an ILR in accordance with the present invention.

FIG. 7 is a flow chart of a method of implanting a subcutaneous ILR according to an embodiment of the present invention.

FIG. 8 is a partial perspective view of an implanting physician utilizing an apparatus to aid in the implant of an ILR in accordance with the present invention.

FIG. 9 is a partial perspective view of an implanting physician utilizing an apparatus to aid in the implant of an ILR in accordance with the present invention.

FIG. 10 is a partial perspective view of an implanting physician utilizing an apparatus to aid in the implant of an ILR in accordance with the present invention.

FIG. 11 is a partial perspective view of an implanting physician utilizing an apparatus to aid in the implant of an ILR in accordance with the present invention.

FIG. 12 is a partial perspective view of an implanting physician utilizing an apparatus to aid in the implant of an ILR in accordance with the present invention.

FIG. 13 is a partial perspective view of an implanting physician utilizing an apparatus to aid in the implant of an ILR in accordance with the present invention.

FIG. 14 is a partial perspective view of an implanting physician utilizing an apparatus to aid in the implant of an ILR in accordance with the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but, on the contrary, the intention is to cover all modifications, equivalents, and alternatives falling within the spirit and scope of the invention as defined by the appended claims.

### DETAILED DESCRIPTION OF THE INVENTION

A small and easy-to-implant, leadless ILR device will require a minimal incision size, which has patient benefit. Between ½ and 1 inch incisions are preferred to avoid trauma and scarring and reduce the chance of infection. For ease of insertion, the device should be easy to self-position, and preferably elongated in shape to maximize signal strength for a given volume by having electrodes spaced at far ends of the length. The general use, without limitation, of the device is long term ECG event monitoring.

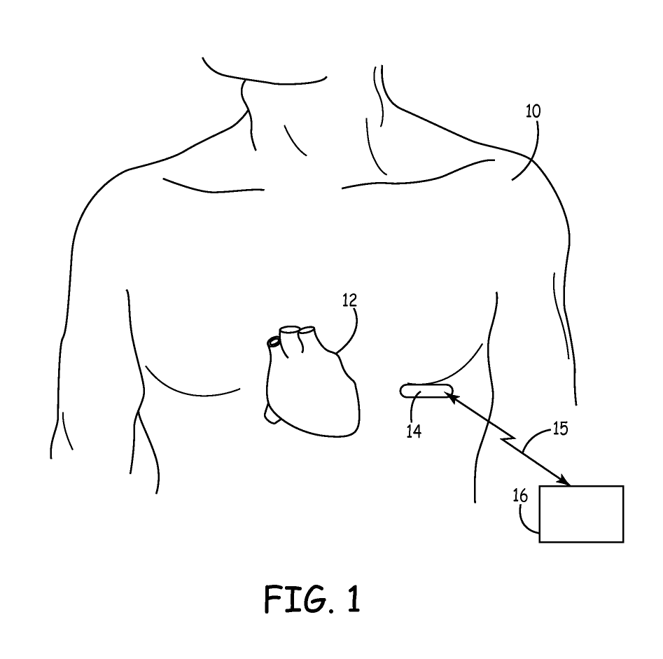

FIG. 1 is a frontal view of a patient 10 in whom an ILR 14 may be subcutaneously implanted with a typical location referenced thereon (other implant locations may be utilized). The ILR 14 senses cardiac electrical activation signals via electrodes (not shown in FIG. 1) from heart 12. A communication link 15 allows 2-way telemetry communication between ILR 14 and an external device (typically a programmer) 16. Programmer 16 and telemetry systems (15) suitable for use in the practice of the present invention have been well known for many years. Known programmers typically communicate with an implanted device via a bi-directional radio-frequency telemetry link, so that the programmer 16 can transmit control commands and operational parameter values to be received by the implanted device 14, and so that the implanted device 14 can communicate captured and stored diagnostic and operational data to the programmer 16. Programmers believed to be suitable for the purposes of practicing the present invention include the Models 9790 and CareLink® programmers, commercially available from Medtronic, Inc., Minneapolis, Minn. Various telemetry systems for providing the necessary communications channels between an external programming unit 16 and an implanted device 14 have been developed and are well known in the art. Telemetry systems believed to be suitable for the purposes of practicing the present invention are disclosed, for example, in the following U.S. patents: U.S. Pat. No. 5,127,404 to Wyborny et al. entitled “Telemetry Format for Implanted Medical Device”; U.S. Pat. No. 4,374,382 to Markowitz entitled “Marker Channel Telemetry System for a Medical Device”; and U.S. Pat. No. 4,556,063 to Thompson et al. entitled “Telemetry System for a Medical Device”. The Wyborny et al. '404, Markowitz '382, and Thompson et al. '063 patents are commonly assigned to the assignee of the present invention, and are each hereby incorporated by reference herein in their respective entireties. With respect to a compact subcutaneous ICD susceptible of benefiting from the present invention U.S. patent application Ser. No. 10/985,341, by Karel F. A. A. Smits entitled “Precordial-Superior Vena Cava Electrode Arrangement For An Implantable Cardioverter Defibrillator” is hereby incorporated in its entirety as if fully set forth herein.

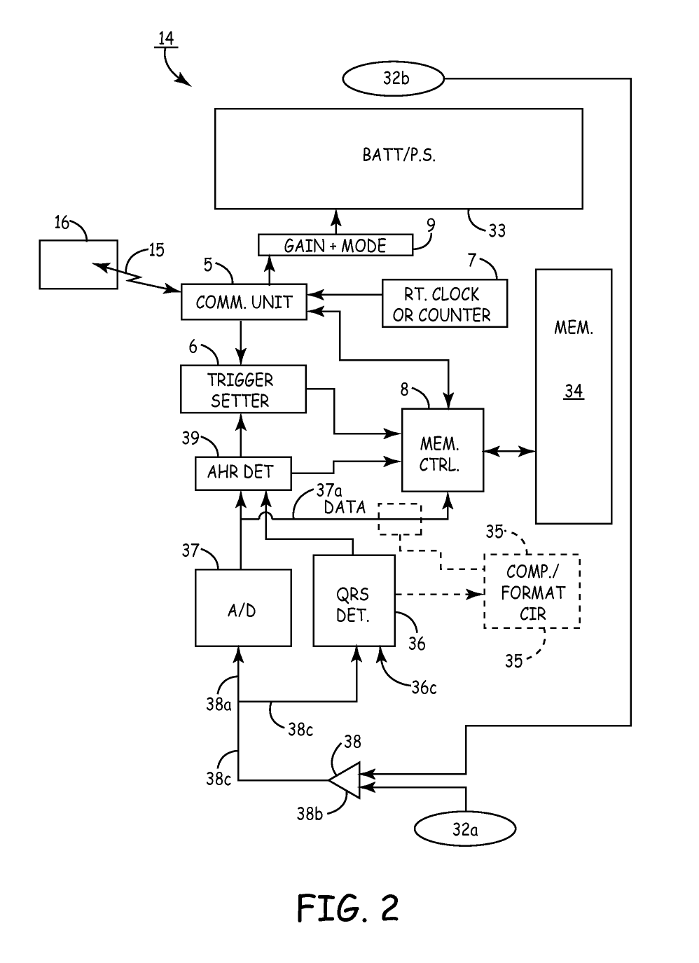

FIG. 2 illustrates the electronic circuitry including low voltage battery 33 within the hermetically sealed housing of ILR 14. The low voltage battery 33 comprises of typically one conventional LiCFₓ, LiMnO₂, or LiI₂ cell. Electrodes 32a and 32b bring a signal from the body to an input mechanism 38, here drawn as a differential amplifier for simplicity only, the output of which is fed to a QRS detector 36 and an A/D converter 37. Both these circuits 36,37 supply an output to an arrhythmia detector 39, which in this preferred embodiment supplies the autotrigger signal to the trigger setting circuit 6. The data output from the A/D converter may be converted, compressed, formatted and marked or reformulated if desired in a circuit 35 before the data is ready for input into the memory 34. The memory control circuit 8 receives input from the A/D converter 37, with or without conversion and so forth from circuit 35, from the auto triggering determination circuit (here seen as the arrhythmia detection circuit) 39 (which may include input directly from the QRS detector if desired) as well as signals from the trigger setter circuit 6. The trigger setter circuit may also be controlled by a communications unit 5 which operates to receive and decode signals from the outside of the implant 14 that are telemetered or otherwise communicated in by a user. This communications unit 5 will also be able to communicate with the memory controller to request the offloading of memory data for analysis by an outside device such as a programmer 16. It should contain an antenna or other transceiver device or circuitry to communicate with an outside device such as programmer 16. A clock or counter circuit 7 reports the time since start or real time to the outside interrogator device 16 contemporaneously with a data offloading session so that the events recorded in memory 34 may be temporally pinpointed.

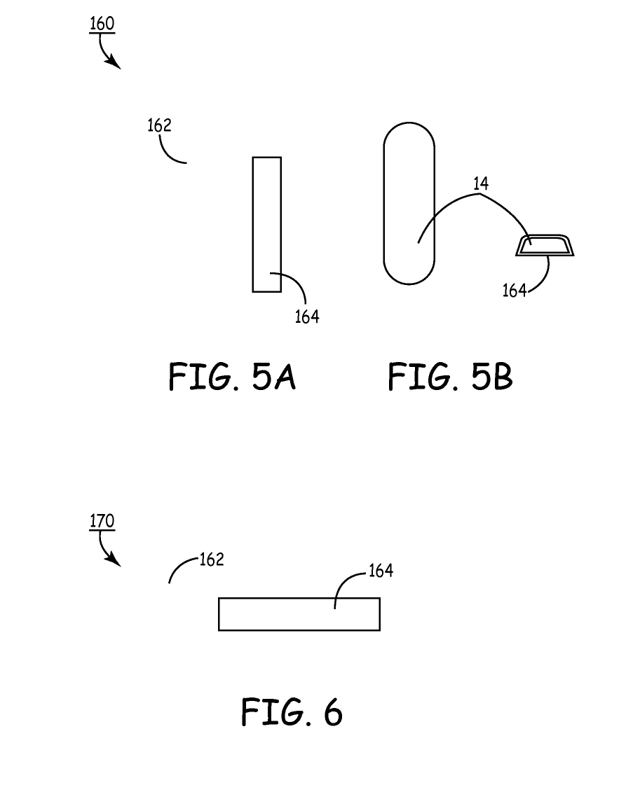

Referring now to FIG. 5B which is a plan view and an elevational side view in cross section) illustrating an ILR 14 in accordance with the present invention. The ILR 14 of the present invention is contained in an extended ovaloid housing with opposing blunt ends for ease of insertion and self-positioning. The extended ovaloid shape maximizes signal strength for a given volume by having electrodes (not shown in FIG. 5B) spaced at far ends of the length. The inferior flat surface of the housing of ILR 14 is larger than the superior surface minimizing the volume of the implanted device for patient comfort during normal torso movement and prevents the patient from “flipping over” the device (i.e., so-called “twiddler syndrome”) preventing the lose of cardiac signal. Additionally, the housing of ILR 14 cross section allows the inventive implant tools of the present invention to deliver the device in the correct orientation subcutaneously, preventing physician error in the implant process and easing and speeding the implant process.

The housing or canister of ILR 14 may be constructed of stainless steel, titanium or ceramic as described in U.S. Pat. No. 4,180,078 “Lead Connector for a Body Implantable Stimulator” to Anderson or U.S. Pat. No. 5,470,345 “Implantable Medical Device with Multi-layered Ceramic Enclosure” to Hassler, et al. The electronics circuitry of ILR 14 (described herein above in relation to FIG. 2) may be incorporated on a polyamide flex circuit, printed circuit board (PCB) or ceramic substrate with integrated circuits packaged in leadless chip carriers, chip scale packaging (CSP) and/or attached to a substrate and glop coated.

The typical dimensions of ILR 14 of the inventive design with an expected 12-14 months longevity are 5.3 mm (length), 1.5 mm (width) and 0.55 mm (height) although a wide variety of dimensions, shapes and sizes of an ILR, or more generally, an IMD benefit from the tools and methods provided per the present invention.

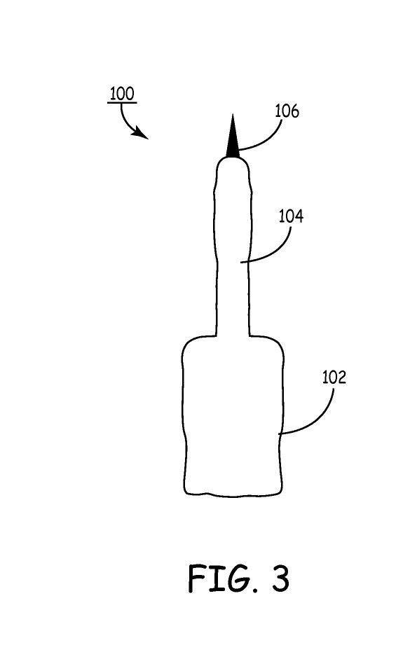

FIG. 3 is a plan view illustrating a poker apparatus to aid in the implant of an ILR 14. The poker 100 is used to make a small initial or starting incisions (e.g., 2 mm) using a pointed portion 106 on the distal portion of poker 100. An exemplary poker 100 includes a handle 102 connected to a shaft 104 with the portion 106 tapered (e.g., from five to 15 degrees of taper). Poker 100 may be constructed out of metal or plastic or any other suitable resilient material. Of course, different portions of the poker 100 can be constructed out of different materials, such as portion 106 comprising a medical grade metal and handle 102 comprising a thermoplastic material.

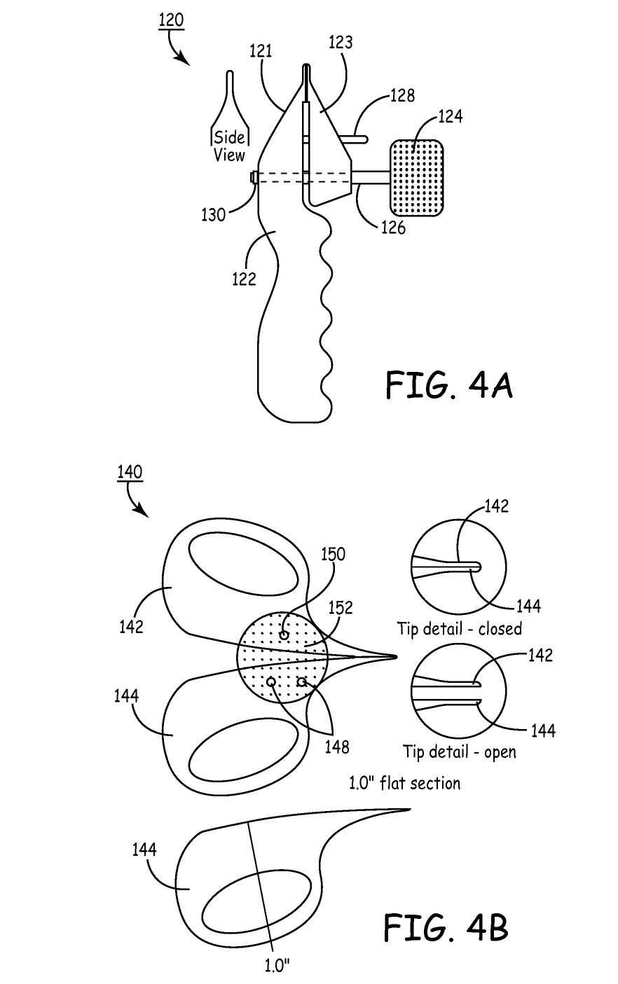

FIG. 4A is a plan view illustrating a tissue spreader apparatus to aid in the implant of an ILR 14 or other subcutaneous IMD. Tissue spreader 120 is used to open the starting hole/incision produced by poker 100. Tissue spreader 120 consists of a handle 122 with a distal tapered tip 121 and an opposing tapered tip 123. The 2 tapered tips (121 and 123) may be spread apart via turning a handle 124 attached to a threaded rod 126 inserted into a like-threaded channel through tapered tip 123. The threaded rod 126 could comprise a quarter-inch (¼″) diameter fine thread (e.g., unified national coarse). A circular 130 clip holds the threaded rod 126 in handle 122. An anti-rotate pin 128 prevents tapered tips 121 and 123 from rotating out of alignment. The handle 122 with tapered tip 121, tapered tip 123 and knurled knob 124 may be constructed of metal (i.e., stainless steel) or of a polymeric material (i.e., a thermoplastic such as the acetyl homopolymer Delrin or other similar polymeric material).

FIG. 4B is a plan view illustrating an alternative tissue spreader apparatus to aid in the implant of an ILR 14. Tissue spreader 140 consists of 2 mirror image scissor-like handles 142 and 144. A round disk 146, fixedly connected to handle 144 by 2 fasteners 148 and a pin 150 located in a slot 152 in handle 142 allow the 2 handles to be squeezed together and forcing the 2 tapered tips of tissue spreader 140 to move from a closed position (FIG. 4B inset 154) to an open position at a fixed gap (FIG. 4B inset 156). The handles 142 and 144 with tapered tips may be constructed of metal (i.e., stainless steel) or of a polymeric material (i.e., a thermoplastic such as the acetyl homopolymer Delrin or other similar polymeric material). Two sizes may be used, a ⅛″ spreader with tips spreading 0.4″ and a ¼″ spreader with tips spreading 0.7″.

FIG. 5A is a plan view illustrating an introducer/cannula apparatus 160 to aid in the implant of an ILR 14. Introducer 162 consists of a handle and an opposing extended tapered end of a similar cross section of the ILR 14 housing. Cannula 164 is a hollow tube of similar, but slightly larger cross section of ILR 14 housing. In use, cannula 164 is slide over the distal end of introducer 162 (as shown in FIG. 6). Cannula 164 and introducer 162 may be constructed out of metal or plastic.

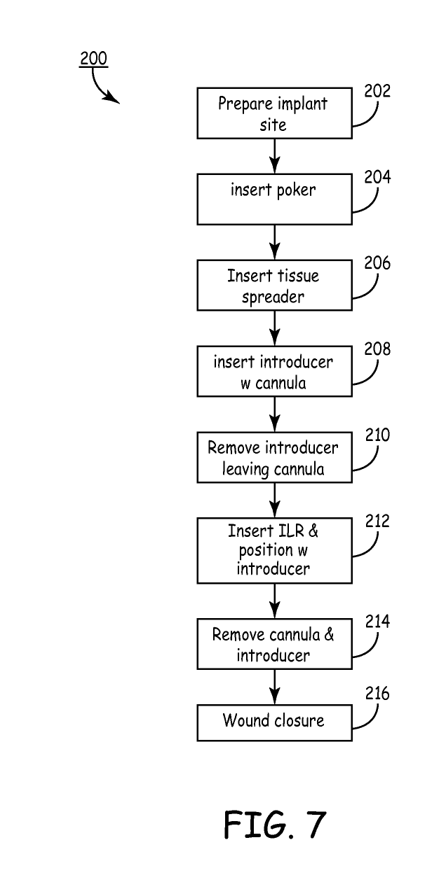

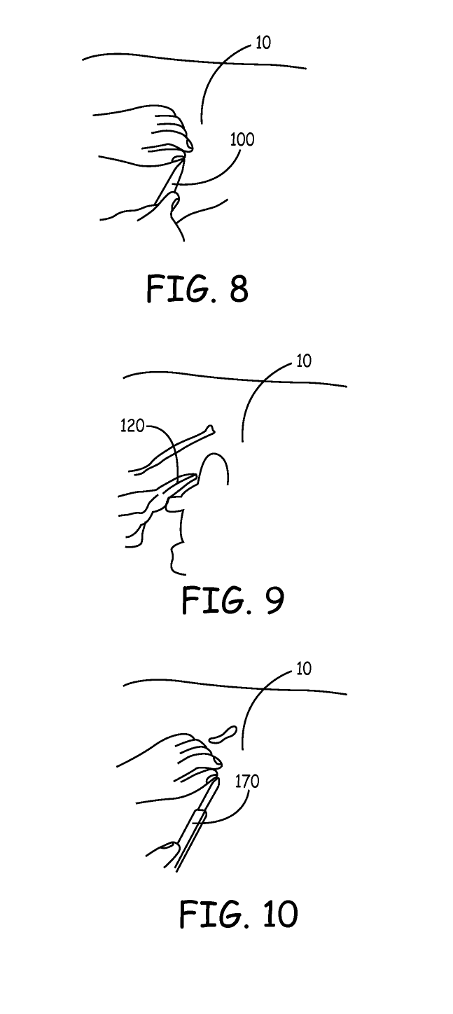

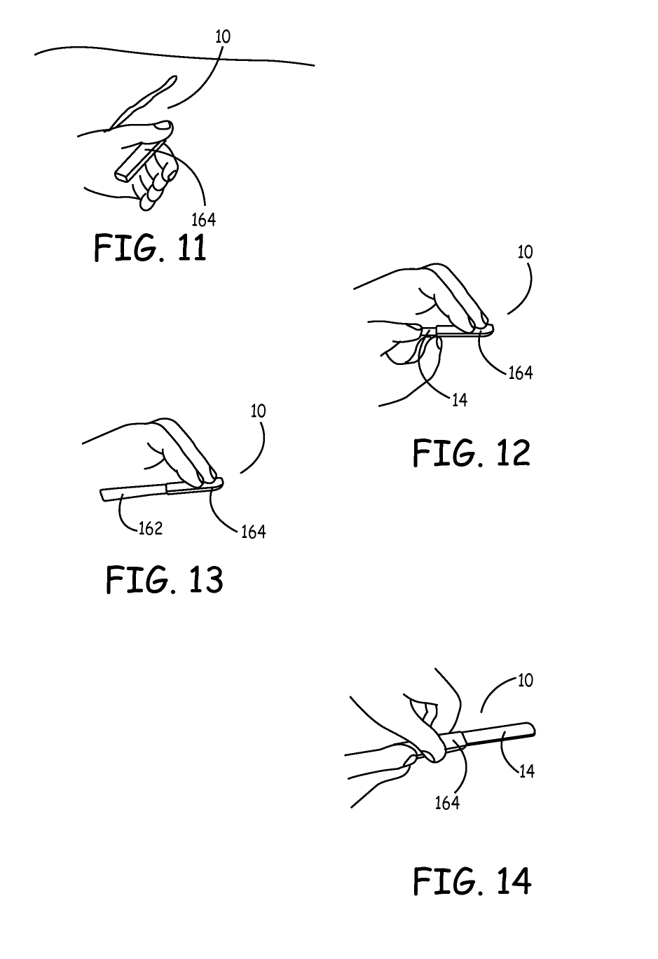

FIG. 7 is a flow chart 200 of a method of implanting a subcutaneous ILR 14 according to an embodiment of the present invention. At step 202 the implant site is prepared (i.e., washed, shaved, sterilized, draped, etc.) per standard surgical procedures. At step 204, the poker 100 is inserted into the patient's skin and generates a 2 mm hole/incision transcutaneously (see FIG. 8). At step 206, tissue spreader 120 (shown FIG. 4A), or alternatively, 140 (shown FIG. 4B) is inserted into the 2 mm incision and operated to increase the incision to 0.7″ width (see FIG. 9). At step 208, the introducer/cannula combination 170 (FIG. 6) is inserted into the incision and, by blunt dissection, a void is generated subcutaneously by the distal blunt end of the introducer/cannula combination (see FIG. 10). At step 210, the introducer 162 is retracted from the cannula 164, leaving the cannula at the entrance to the incision (see FIG. 11). At step 212, the ILR 14 is inserted into the cannula 164 (see FIG. 12), pushed through the cannula and into the subcutaneous implant site by the introducer 162 (see FIG. 13). At step 214, the introducer 162 and cannula 164 are removed leaving the ILR 14 in a subcutaneous implant site (see FIG. 14). At step 216, the incision is closed by any of several methods commonly used for small incisions/wounds (e.g., steri-strips, wound glue, sutures and/or clips).

The above described inventive apparatus and process is completed with simple tools and processes without requiring expensive surgical suites, procedures, scalpels, cautery machines, and the like. It thus lends itself to an outpatient process by less skilled physicians and fewer supporting staff.

It will be apparent from the foregoing that while particular embodiments of the invention have been illustrated and described, various modifications can be made without departing from the spirit and scope of the invention. Accordingly, it is not intended that the invention be limited to the depicted embodiments but rather be interpreted broadly in accordance with a reasonable interpretation of the appended claims.

1. A kit for implanting a subcutaneous implantable medical device (IMD) without use of a scalpel, comprising:
a manipulable instrument having a pointed distal end, said manipulable instrument adapted to pierce a portion of epidermis and dermis of a subject;
a tissue spreading instrument configured to deploy into said pierced portion of epidermis and dermis of the subject, wherein said tissue spreading instrument includes opposing distal tip portions and wherein said distal tip portions couple to structure and have a range of motion therebetween
an elongated hollow introducer instrument adapted for insertion into said pierced portion of epidermis and dermis of the subject; and
a hollow cannula adapted for slideable insertion into the elongated hollow introducer instrument.

2. A kit according to claim 1, further comprising a wound closure element.

3. A kit according to claim 2, wherein the wound closure element consists of at least one of the following from the group consisting of:
a sterile adhesive strip, a volume of wound closure adhesive, a mechanical clip, a segment of suture, a mechanical staple.

4. A kit according to claim 1, wherein the pointed distal end portion includes a base portion having a diameter of approximately two millimeters.

5. A kit according to claim 1 wherein the tissue spreading instrument comprises one of a scissor-type tissue spreader and a threaded-type tissue spreader.

6. A kit according to claim 1, wherein the tissue spreading instrument includes a mechanical stop providing for a predetermined amount of separation between the opposing distal tip portions.

7. A kit according to claim 6, wherein the predetermined amount of separation comprises a range of between approximately one-half inch (½″) and one inch (1″).

8. A kit according to claim 1, wherein the IMD comprises one of a subcutaneous implantable loop recorder (ILR), an implantable pulse generator (IPG), and a subcutaneous implantable cardioverter-defibrillator (ICD).

9. A kit according to claim 1, wherein a housing for the IMD includes a relatively uniform cross-sectional shape and wherein the interior of at least one of said elongated hollow introducer instrument and said hollow cannula closely approximate the relatively uniform cross-sectional shape of said IMD.

10. A kit according to claim 9, wherein the relatively uniform cross-sectional shape of the IMD comprises at least one of a relatively uniformly tapered cross-sectional shape and an ovoid shape.

11. A kit according to claim 1, wherein said components of said kit are all disposed in a single sterile package, and wherein said package includes indicia regarding one of: manufacturing date, manufacturer identification, approved implantation jurisdiction(s), expiration date.

12. A method of implanting a self-contained subcutaneous implantable medical device (IMD) without use of a scalpel, comprising:
preparing a subcutaneous implantation site;
inserting a manipulable instrument having a pointed distal end into a portion of epidermis and dermis of a subject within said implantation site;
removing said manipulable instrument from said insertion location;
spreading tissue adjacent the insertion location with a tissue spreading instrument configured to deploy into said insertion location, wherein said tissue spreading instrument includes opposing distal tip portions and wherein said distal tip portions couple to structure and have a range of motion therebetween;
removing said tissue spreading instrument from said insertion location;
inserting an IMD into a hollow cannula;
inserting an elongated hollow introducer instrument having the hollow cannula disposed within the introducer instrument into said spread tissue adjacent the insertion location; and
deploying the IMD into said insertion location.

13. A method according to claim 12, further comprising removing said elongated hollow introducer and said hollow cannula from the insertion location.

14. A method according to claim 12, wherein the IMD comprises one of a subcutaneous implantable loop recorder (ILR), an implantable pulse generator (IPG), and a subcutaneous implantable cardioverter-defibrillator (ICD).

15. A method according to claim 12, wherein a housing for the IMD includes a relatively uniform cross-sectional shape and wherein the interior of at least one of said elongated hollow introducer instrument and said hollow cannula closely approximate the relatively uniform cross-sectional shape of said IMD.

16. A method according to claim 12, further comprising:
closing the spread tissue by applying a wound closure element.

17. A method according to claim 13, wherein the wound closure element comprises at least one of the following from the group consisting of: a sterile adhesive strip, a volume of wound closure adhesive, a mechanical clip, a segment of suture, a mechanical staple.
